(19) United States
(12) Patent Application Publication
(10) Pub. No.: US 2015/0265075 A1
(43) Pub. Date: Sep. 24, 2015

(54) SNORE SUPPRESSION PILLOW

(71) Applicant: GREEN-SWEET MATTRESS CORP., New Taipei City (TW)

(72) Inventors: Chih-Yuan Liu, New Taipei City (TW); Wen-Chyuan Chen, New Taipei City (TW); Hsueh-Yu Li, New Taipei City (TW); Li-Ang Lee, New Taipei City (TW)

(21) Appl. No.: 14/641,437

(22) Filed: Mar. 9, 2015

(30) Foreign Application Priority Data
Mar. 19, 2014 (TW) 103204682

(51) Int. Cl. A47G 9/10; A61F 5/56

(52) U.S. Cl. CPC A47G 9/10; A61F 5/56

(57) ABSTRACT

A snore suppression pillow includes a pillow body and at least one raising layer. The pillow body has a surface forming a high end and a low end. The high end includes a recessed portion formed in each of two sides of a middle portion thereof so that the middle portion is configured as a curved protrusion. The at least one raising layer is attachable to a bottom of the pillow body. As such, adjustment is achievable with the at least one raising layer to provide the pillow with a height suiting the need of a user and the protrusion of the pillow body allows the head of the user to turn aside to the left side or right side when the user is sleeping so as to stretch the respiratory tract to help prevent snoring caused by slackening of the respiratory tract.

### TECHNICAL FIELD OF THE INVENTION

The present invention relates generally to a snore suppression pillow, and more particularly to a pillow that is particularly suitable for sleep in for example home, hotels, or similar facility.

### DESCRIPTION OF THE PRIOR ART

The main cause of snoring is position change of some collapsible structures of the upper respiratory tract, such as the uvula, the soft palate, anterior and posterior arches of the tonsil, and root of the tongue, which leads narrowing of the respiratory tract and sound caused by vibration inhalation and exhalation of air through the respiratory tract is thus generated. Such a symptom, for a not very serious condition, will affect the sleep of other people, and for a serious condition, may cause interruption of breathing during sleeping and in the worst case, leading to death.

For such problems, the present inventor has proposed, in Taiwan Utility Model M446586, a snoring prevention pillow, which is helpful in improving the symptom of a snorer and is generally composed of an integrally formed pillow body over which a pillowcase is fit and is characterized in that the pillow body has an upper surface that forms a raised portion. The raised portion comprises a curved protrusion occupying a portion of the upper surface of the pillow body and the height, the area, and the shape of the raised portion correspond to and support the neck of a user who lies down on bed so that when the user falls asleep, the raised portion that provides a support that is small in size causes the head of the user to turn sideways to the right or left side.

Such a known structure, although showing an advantage of effectively preventing snoring, is made in a unitary form, making it not possible to adjust the height thereof. Thus, further improvement is desired.

In view of these problems, the present invention aims to provide a snore suppression pillow that helps change the sleeping posture of a user to stretch the respiratory tract in such a way as to prevent the sound caused by vibration due to slackening of the respiratory tract.

### SUMMARY OF THE INVENTION

The primary object of the present invention is to provide a snore suppression pillow that helps stretch the respiratory tract of user by changing the sleeping posture so as to prevent the sound caused by vibration due to slackening of the respiratory tract.

To achieve the above object, the present invention provides a snore suppression pillow, which comprises a pillow body and at least one raising layer. The pillow body has a surface forming a high end and a low end. The high end comprises a recessed portion formed at each of two sides of a middle portion thereof so that the middle portion forms a curved protrusion.

The at least one a raising layer is attached to a bottom of the pillow body. As such, adjustment is achievable with the at least one raising layer to provide the pillow with a height suiting the need of a user and the protrusion of the pillow body allows the head of the user to turn aside to the left side or right side when the user is sleeping so as to stretch the respiratory tract to help prevent snoring caused by slackening of the respiratory tract.

In the above-described snore suppression pillow, the surface of the pillow body comprises a trough formed in each of two sides of the protrusion.

In the above-described snore suppression pillow, the pillow body comprises a two-layered structure having an outer layer and an inner layer. The outer layer is made of a soft or inertial foamed material. The inner layer is made of a stiff polyurethane (PU) latex or foamed material having predetermined resiliency.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of an embodiment of the present invention.

FIG. 2 is an exploded view of the embodiment of the present invention.

FIG. 3 is a schematic view illustrating the use of the embodiment of the present invention.

FIG. 4 is a schematic view similar to FIG. 3 but illustrating another way of use of the embodiment of the present invention.

FIG. 5 is a schematic view illustrating how snoring is suppressed in the way of use shown in FIG. 4.

FIG. 6 is an exploded view of a second embodiment of the present invention.

FIG. 7 is a schematic view illustrating the use of the embodiment of FIG. 6.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

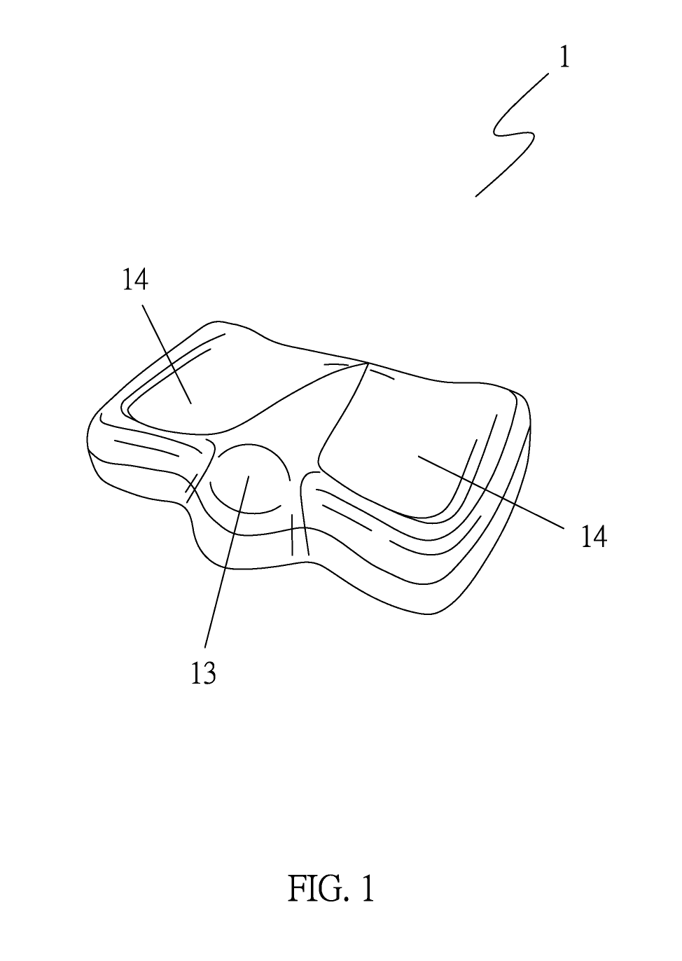

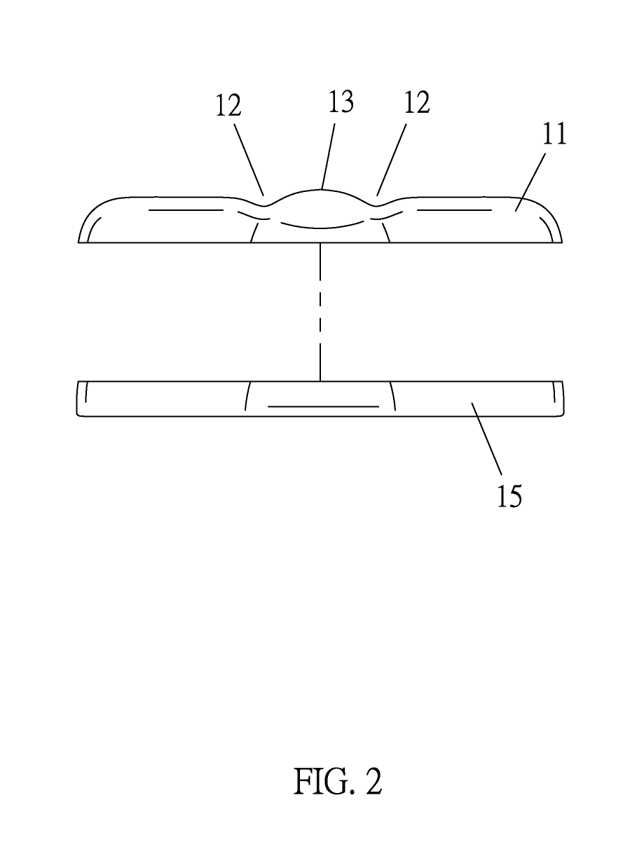

Referring to FIGS. 1 and 2, the present invention provides a snore suppression pillow 1, which comprises a pillow body 11 and at least one raising layer 15. The pillow body 11 has a surface that forms a high end and a low end. The high end comprises a recessed portion 12 formed in each side of a middle portion thereof so that the middle portion is configured as a curved protrusion 13. The surface of the pillow body 11 also comprises two troughs 14 formed therein. The at least one raising layer 15 is attached to a bottom of the pillow body 11. As such, a complete snore suppression pillow 1 is formed.

The pillow body 11 is of a two-layered structure comprising an inner layer and an outer layer. The outer layer is made of a soft and inertial material, such as a foamed material and the inner layer is made of a stiff material that is resilient to some extents, such as polyurethane (PU) rubber latex or a stiff foamed material. The depth of the recessed portion 12 is selected so that the cervical vertebra portion of a user 5 may be supported when the user lies down and sleeps, whereby the protrusion 13 so formed provides a support that is small so that the head of the user 5 is caused to turn aside to the left or right side.

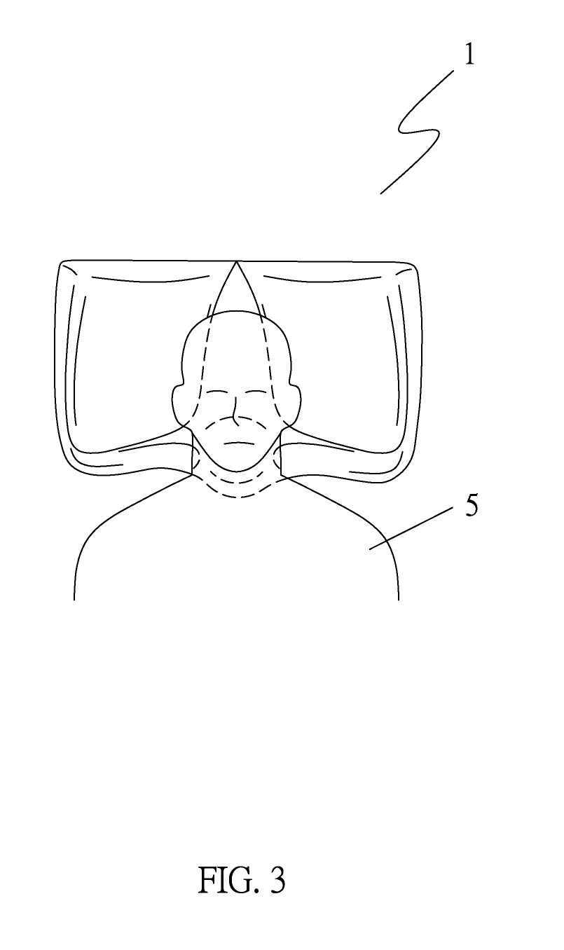

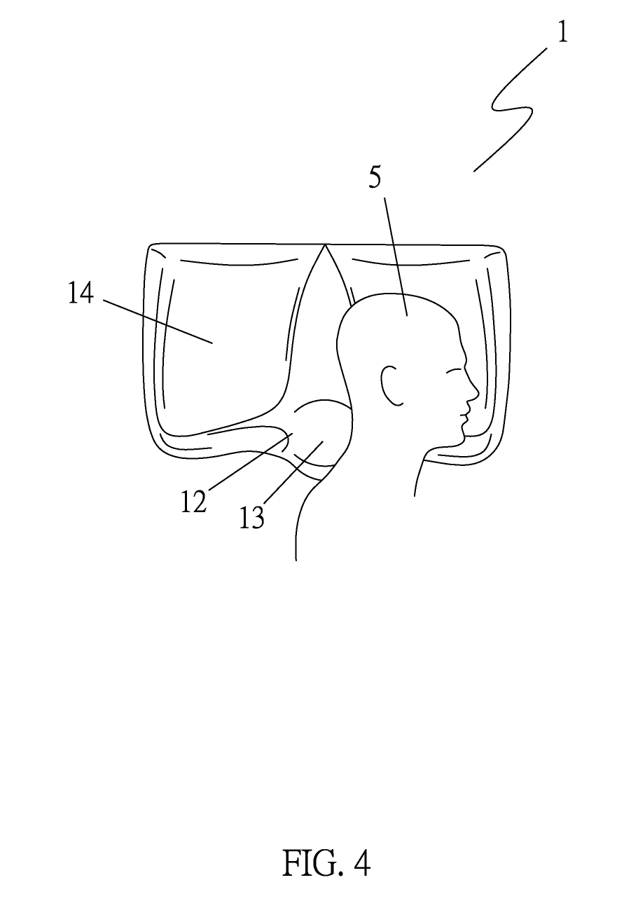

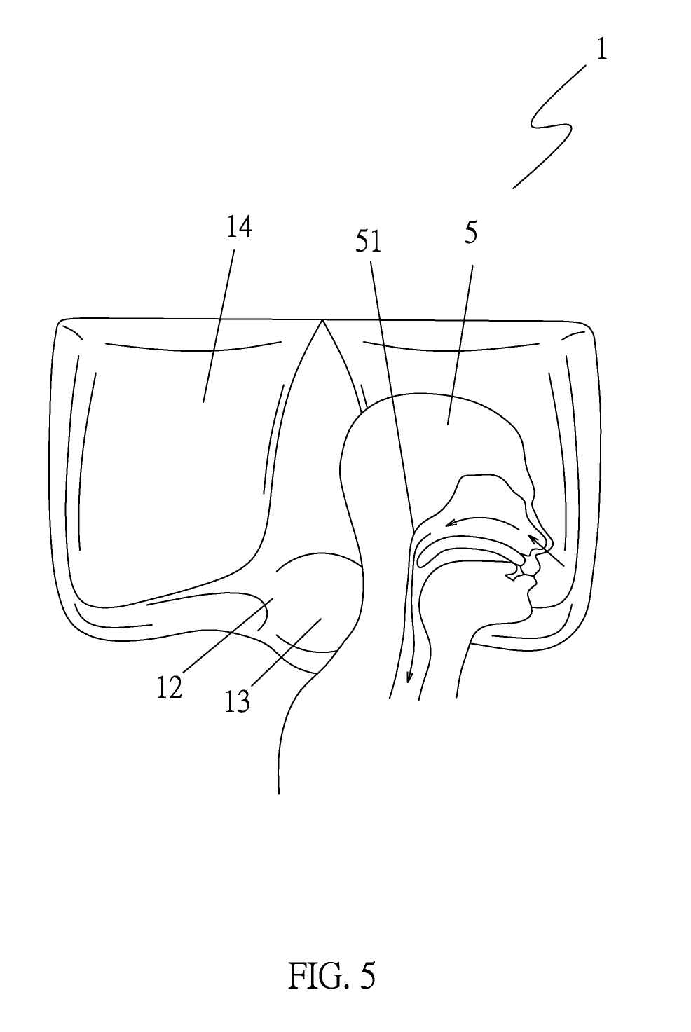

To use, as shown in FIG. 3, the user 5 lies down and sleeps with the cervical vertebra in alignment with the protrusion 13. When just lying down, the user 5 is still conscious so that the cervical vertebra portion can be stably set on the protrusion 13 with the front of the head facing upward. When the user 5 gets into a deep sleep and becomes unconscious, muscles around the cervical vertebra get relaxed so that due to the support point provided by the protrusion 13 is small, the head is turned aside to the left or right side as shown in FIG. 4, where the front of the head faces leftward or rightward, so that the respiratory tract 51 of the user 5 is stretched and thus wide open as shown in FIG. 5 thereby preventing sounding caused by vibration resulting from slackening of the respiratory tract. As such, the purpose of suppressing snoring can be successfully achieved.

Further, to use the present invention in an alternative way, the user 5 may have the head lying sideways in one of the troughs 14 to show a sideway sleeping posture, in such a condition, no snoring that is caused by the respiratory tract getting narrowed resulting from excessively released may occur. Further, the raising layer 15 that is stacked under the pillow body 11 may be increased or decreased in the number thereof to suit personal need of the user so that an optimum height may be obtained through the adjustment.

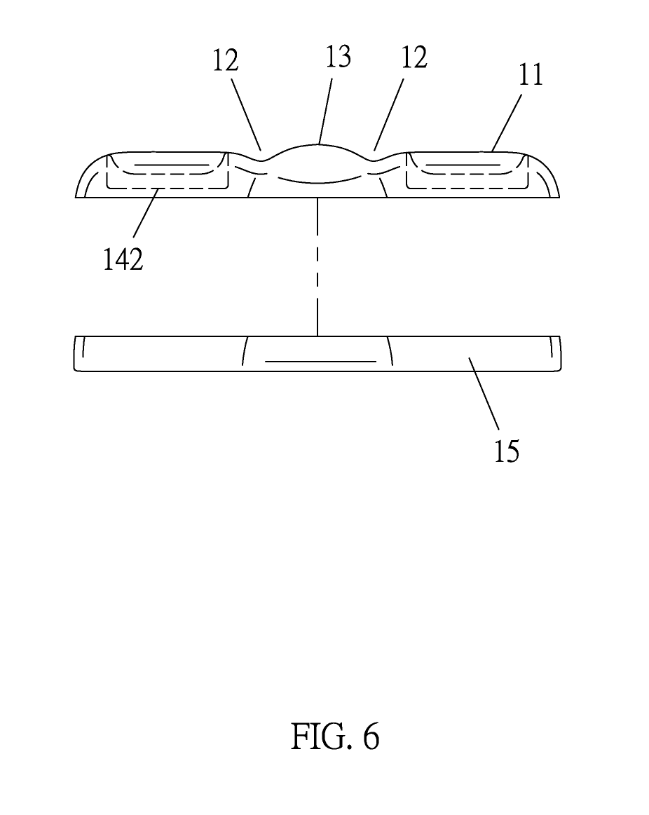

Referring to FIG. 6, a snore suppression pillow 1 according to a second embodiment of the present invention is shown. The pillow body 11 is of a two-layered structure comprising an outer layer and an inner layer. The outer layer is made of a soft or inertial foamed material, while the inner layer is made of a stiff PU rubber latex or foamed material that shows resiliency to some extents.

In the instant embodiment, the troughs 14 of the pillow body 11 each comprise a first zone 141 and a second zone 142. The second zone 142 comprises a foamed material of which the elastic coefficient is greater than that of the foamed material of the outer layer so that softness can be enhanced.

The second zone 142 is integrally formed with the pillow body 11, or alternatively, the second zone 142 is a detachable piece of foamed material that is coupled to the pillow body 11 through fitting.

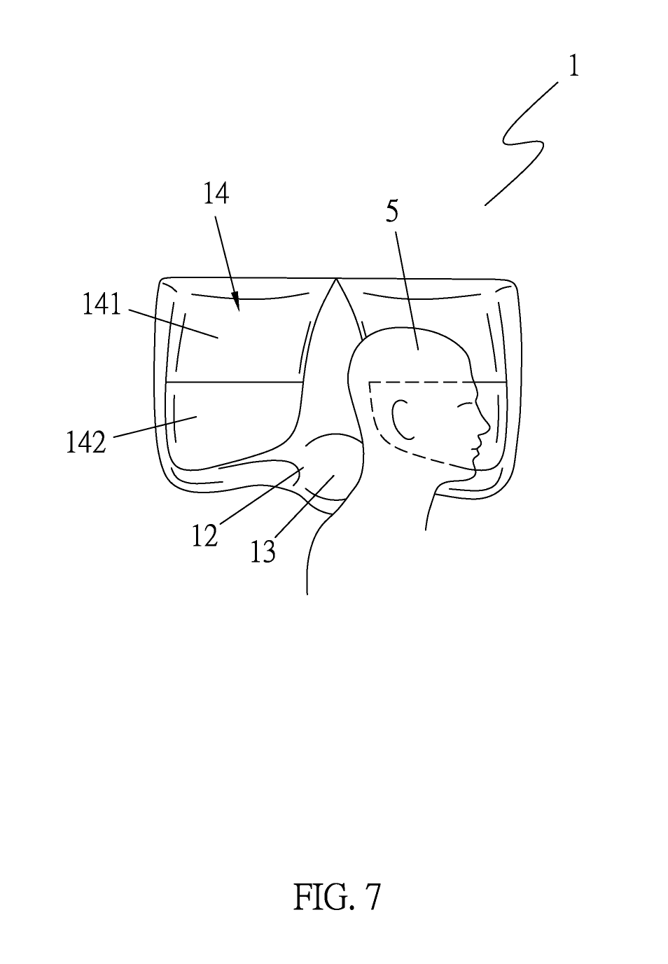

Referring to FIG. 7, when a person is sleeping in a sideway posture, one ear bears a relatively great force and headache or sleeping disorder may occur for a long term of bearing the force. Thus, the second zone 142 of the pillow body 11 is structured to include a foamed material of which the elastic constant is greater than that of the outer layer for the purposes of reducing the force applied to the ear to eventually enhance sleep quality

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A snore suppression pillow, comprising a pillow body and at least one raising layer, the pillow body having a surface forming a high end and a low end, the high end comprising a recessed portion formed at each of two sides of a middle thereof so that the middle forms a curved protrusion, the at least one a raising layer being attached to a bottom of the pillow body, wherein adjustment is achievable with the at least one raising layer to provide the pillow with a height suiting the need of a user and the protrusion of the pillow body allows the head of the user to turn aside to the left side or right side when the user is sleeping so as to stretch the respiratory tract to help prevent snoring caused by slackening of the respiratory tract.

2. The snore suppression pillow according to claim 1, wherein the surface of the pillow body comprises a trough formed in each of two sides of the protrusion.

3. The snore suppression pillow according to claim 1, wherein the pillow body comprises a two-layered structure having an outer layer and an inner layer, the outer layer being made of a soft or inertial foamed material, the inner layer being made of a stiff polyurethane (PU) latex or foamed material having predetermined resiliency.

4. The snore suppression pillow according to claim 1, wherein the troughs of the pillow body each comprise a first zone and a second zone, the second zone comprising a foamed material of which elastic coefficient is greater than that of the outer layer.

5. The snore suppression pillow according to claim 4, wherein the pillow body and the second zone are integrally formed together.

6. The snore suppression pillow according to claim 4, wherein the second zone comprises a detachable piece of foamed material.
